(19) United States
(12) Patent Application Publication
(10) Pub. No.: US 2021/0187128 A1
(43) Pub. Date: Jun. 24, 2021

(54) COLOR CHANGING SILK PATCH FOR VISIBLE ROS DETECTION

(71) Applicants: University of Central Florida Research Foundation, Inc., Orlando, FL (US); The Regents of the University of Colorado, a Body Corporate, Denver, CO (US)

(72) Inventors: Sudipta Seal, Orlando, FL (US); Sushant Singh, Orlando, FL (US); Kenneth W. Liechty, Aurora, CO (US); Carlos Zgheib, Aurora, CO (US)

(21) Appl. No.: 17/131,956

(22) Filed: Dec. 23, 2020

Related U.S. Application Data
(60) Provisional application No. 62/953,227, filed on Dec. 24, 2019.

(51) Int. Cl. A61K 49/00; A61K 47/42; A61K 9/70

(52) U.S. Cl. CPC A61K 49/0063; A61K 9/7007; A61K 47/42; A61K 49/0028

(57) ABSTRACT

An electrospun nanofibrous silk fibroin mat infused with a reactive oxygen species (ROS) detection reagent is presented herein. The ROS detection agent may be Amplex red. A method of real time detection of ROS in a wound site to determine the need for a dressing change is also presented. The method includes applying an electrospun nanofibrous silk fibroin mat infused with Amplex red to a wound site of a patient and detecting a change in color of the mat. A color change indicates a higher than normal level of ROS in the wound site. Depending on the degree of color change, a therapeutic agent may be administered to the wound site and a new mat applied to measure ROS in the wound site after therapy is administered.

### CROSS REFERENCE TO RELATED APPLICATIONS

This application is a nonprovisional of and claims priority to U.S. Provisional Application No. 62/953,227, entitled “A Color Changing Silk Patch for Visible ROS Detection”, filed Dec. 24, 2019, the contents of which are hereby incorporated by reference into this disclosure.

### FIELD OF THE INVENTION

This invention relates to wound care. More particularly, it relates to a silk fibroin mat capable of visually sensing oxidative stress in cutaneous wounds.

### BACKGROUND OF THE INVENTION

Cutaneous wounds on the skin have always been of major concern in clinical practice worldwide. (G. Han and R. Ceilley, Adv. Ther., 2017, 34, 599-610). The advancement of nanomedicine technology has constantly been explored for determining efficient methods for restoring damaged tissues integrity and function and promote healing. (G. Sener, S. A. Hilton, M. J. Osmond, C. Zgheib, J. P. Newsom, L. Dewberry, S. Singh, T. S. Sakthivel, S. Seal, K. W. Liechty and M. D. Krebs, Acta Biomater., 2020, 101, 262-272; M. B. Dreifke, A. A. Jayasuriya and A. C. Jayasuriya, Mater. Sci. Eng., C, 2015, 48, 651-662).

Human skin serves as a protective barrier against the harsh external environmental aggressors. In case of dermal wounds, its condition is compromised and the ability to work as a protective barrier is highly impaired. Under the damaging conditions of the cutaneous wounds, the immune system initiates a cascade of protective natural mechanisms to correct this impairment and efficient healing of the wound. Normal wound repair follows an orderly and well-defined sequence of events that requires the interaction of many cell types and growth factors, and is divided into 3 main phases: inflammatory, proliferative, and remodeling phases. (H. Wu, F. Li, S. Wang, J. Lu, J. Li, Y. Du, X. Sun, X. Chen, J. Gao and D. Ling, Biomaterials, 2018, 151, 66-77).

At the site of the wound, during an early inflammatory response, the inflammatory cells such as neutrophils and macrophages are highly recruited to the site of injury and destroy potential pathogen by phagocytosis and the productions and release of antimicrobial peptides, proteases and reactive oxygen species (ROS). (H. Steiling, B. Munz, S. Werner and M. Brauchle, Exp. Cell Res., 1999, 247, 484-494; T. A. Wilgus, S. Roy and J. C. McDaniel, Adv. Wound Care, 2013, 2, 379-388). ROS are the by-products of oxygen metabolism and are produced by a variety of cells at the site of inflammation such as platelets, white blood cells, and the mitochondria. The ROS are capable of oxidative killing of bacteria that infiltrate the wound arena. The different ROS molecules produced are superoxide dismutase (O²⁻), hydrogen peroxide (H₂O₂), hydroxyl radical (.OH), peroxy nitrate (ONOO⁻), that are are neutralized by the cellular antioxidant molecules like superoxide dismutase (SOD), catalase (CAT), and peroxidases within the cellular system. (T. Pirmohamed, J. M. Dowding, S. Singh, B. Wasserman, E. Heckert, A. S. Karakoti, J. E. King, S. Seal and W. T. Self, Chem. Commun., 2010, 46, 2736-2738).

The ROS molecules also play an important role in cellular signaling and initial wound healing. ROS activity stimulates the cytokinin and chemokine-receptor activation and the hypoxia induce the cytokine release. (H. M. Kimmel, A. Grant and J. Ditata, Wounds, 2016, 28, 264-270). Overall, the combination of these effects attracts the major components of the wound healing as a part of the immune systems defense mechanism against invading pathogens. However, in certain cases where normal wound healing is impaired, such as diabetic wounds, the excess amount of ROS can cause damage to proteins, DNA, lipids, and carbohydrates as well as inducing endothelial dysfunctions (i.e. cellular senescence and fibrotic scarring and further tissue damage. This process is overall referred to as oxidative stress condition. (T. Pirmohamed, J. M. Dowding, S. Singh, B. Wasserman, E. Heckert, A. S. Karakoti, J. E. King, S. Seal and W. T. Self, Chem. Commun., 2010, 46, 2736-2738; M. Mittal, M. R. Siddiqui, K. Tran, S. P. Reddy and A. B. Malik, Antioxid. Redox Signaling, 2014, 20, 1126-1167; P. D. Ray, B.-W. Huang and Y. Tsuji, Cell. Signalling, 2012, 24, 981-990). Oxidative stress has been implicated with numerous diseases including Parkinson's and Alzheimer's disease, cardiovascular disease, cancer and wounds. (D. J. Betteridge, Metabolism, 2000, 49, 3-8).

The wound environment must be accounted for in order to ensure proper healing of the wound. Current solutions generally include developing therapeutic regimens to correct and enhance wound healing, however little or no consideration is given for monitoring the wound environment and systemic limitations such as ROS levels, which affects how fast these wounds will heal. (C. K. Sen and S. Roy, Biochim Biophys. Acta, Gen. Subj., 2008, 1780, 1348-1361). The ROS levels are measured in vitro using the cultured cells or in vivo by collecting the wound exudates or fluid. (X. Wang, M. K. Sng, S. Foo, H. C. Chong, W. L. Lee, M. B. Y. Tang, K. W. Ng, B. Luo, C. Choong and M. T. C. Wong, J. Controlled Release, 2015, 197, 138-147).

Microplate readers and flow cytometers are generally employed for estimating the amount of ROS in these fluids, and although they provide accurate measurements of these levels, they are not capable of providing the real-time status of the ROS level within the wound environment. (J.-I. Jun and L. F. Lau, Nat. Cell Biol., 2010, 12, 676; E. Aliyev, U. Sakalhoglu, Z. Eren and G. Acikgoz, Biomaterials, 2004, 25, 4633-4637; Y.-H. Lee, J.-J. Chang, C.-T. Chien, M.-C. Yang and H.-F. Chien, Exp. Diabetes Res., 2012, 2012, 504693). Electron paramagnetic resonance (EPR) spin trapping spectroscopy is considered the gold standard for measuring the oxygen-based ROS molecules in a biological system. However, limitations include the formation of EPR silent products when ROS are measured in cells and tissues, leading the failure to detect low level of ROS generation. In light of the foregoing challenges in the current state of the technology, what are needed are new methods of reagents for detecting ROS at the wound site.

### SUMMARY OF INVENTION

Disclosed are methods and compositions related to detection of reactive oxygen species (ROS) at a wound site.

In an embodiment, a composition for detecting reactive oxygen species (ROS) at a wound site is presented comprising: an electrospun nanofibrous mat; and a reactive oxygen species (ROS) reactive detectable reagent infused into the electrospun nanofibrous mat. The nanofibers may be silk obtained from silkworms. The ROS reactive detectable reagent may be an Amplex red compound. In some embodiments, the electrospun nanofibrous mat may be a component of an adhesive bandage, wound dressing, surgical drape, or suture. The electrospun fibrous mat may have a thickness between about 0.001 mm to about 0.0043 mm.

In another embodiment, a method of detecting reactive oxygen species (ROS) levels at a wound site of a patient in real time to determine the need for a dressing change is presented comprising: applying an electrospun nanofibrous mat infused with an ROS detectable reagent to the wound site of the patient; applying an electrospun nanofibrous mat infused with an ROS detectable reagent to the wound site of the patient; and detecting a change in color of the electrospun nanofibrous mat wherein the change in color indicates presence of ROS. The nanofibers may be silk and the ROS detectable reagent may be an Amplex red compound.

In some embodiments, the method further comprises comparing the degree of color change in the electropsun nanofibrous mat to a control to measure an amount of ROS at the wound site of the patient. The intensity of the degree of color change as compared to the control may indicate a high amount of the ROS at the wound site. If an intense degree of color change is detected as compared to the control, a therapeutically effective amount of a therapeutic agent for wound healing may be administered to the wound site.

The electrospun nanofibrous mat may be a singular mat only or alternatively may be a component of an adhesive bandage, wound dressing, surgical drape, or suture.

In a further embodiment, a method of monitoring healing of a wound site in a patient is presented comprising: applying a first electrospun nanofibrous mat infused with an ROS detectable reagent to the wound site of the patient; detecting a change in color of the first electrospun nanofibrous mat; removing the first electrospun nanofibrous mat from the wound site; applying a second electrospun nanofibrous mat infused with an ROS detectable reagent to the wound site of the patient; detecting a change in color of the second electropsun nanofibrous mat; and comparing the change in color of the second electrospun nanofibrous mat to the change in color of the first electrospun nanofibrous mat wherein if the degree of the change in color of the second electrospun nanofibrous mat is less than the degree of the change in color of the first electrospun nanofibrous mat then the wound site is healing.

In some embodiments, a therapeutic agent is administered to the wound site of the patient prior to applying the second electrospun nanofibrous mat.

The nanofibers may be silk and the ROS detectable reagent may be an Amplex red compound. The electrospun nanofibrous mat may be a singular mat only or alternatively may be a component of an adhesive bandage, wound dressing, surgical drape, or suture.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a fuller understanding of the invention, reference should be made to the following detailed description, taken in connection with the accompanying drawings, in which:

FIG. 1 is a schematic representation of oxidation of Amplex Red in the presence of peroxidase and H₂O₂ into a visible color product. Interaction of Amplex Red reagent with H₂O₂ in presence of peroxidase leads to its oxidation into a visible pink colored compound resorufin. Here, this reaction is presented where the vial containing Amplex red and peroxidase interaction remains colorless. However, the addition of H₂O₂ leads to the instantaneous oxidation of Amplex Red into a highly visible pink colored resorufin compound.

FIG. 2 is a schematic representation of the silk fibroin preparation from raw silk cocoons through the degumming process and solubilization. Electrospinning of the silk fibroin solution leads into the formation of nanofibrous silk fibroin mats.

FIG. 3A is a graph depicting the degummed and solubilized silk fibroin solution was also subjected to physiochemical characterization prior to its electrospinning into nanofibrous silk mat. (A) shows dynamic light scattering (DLS) for silk nanofibrous size analysis was performed using this silk fibroin solution. The DLS analysis reveals the average silk nanofibrous size to be in the range of 4 nm. This confirms the solubilization of degummed silk into fine nanofibrous fibers which are essentially required for the nanofibrous silk mat formation.

FIG. 3B is a graph depicting the degummed and solubilized silk fibroin solution was also subjected to physiochemical characterization prior to its electrospinning into nanofibrous silk mat. (B) shows that this graph depicts the average zeta potential analysis of nanofibrous silk solution which is determined to be in the range of −8 mV indicating the silk solution to be non-aggregated and suitable for further electrospinning into nanofibrous silk mat.

FIG. 4A is an image depicting electrospun silk fibroin nanofibrous mats with and without Amplex were characterized using scanning electron microscope (SEM). SEM analysis indicates the formation of continuous silk fibroin nanofibers leading to a synthesis of electrospun silk fibroin mats. (A) represents the nanofibrous silk fibroin mats.

FIG. 4B is an image depicting electrospun silk fibroin nanofibrous mats with and without Amplex were characterized using scanning electron microscope (SEM). SEM analysis indicates the formation of continuous silk fibroin nanofibers leading to a synthesis of electrospun silk fibroin mats. (B) represents nanofibrous silk fibroin mats infused with Amplex red. Formation of ultra-fine continuous nanofibers can be observed in both of these silk fibroin mats.

FIG. 4C is an image depicting electrospun silk fibroin nanofibrous mats with and without Amplex were characterized using scanning electron microscope (SEM). SEM analysis indicates the formation of continuous silk fibroin nanofibers leading to a synthesis of electrospun silk fibroin mats. (C) shows a bar graph of the nanofiber size measurement (from SEM images) using ImageJ analysis of the 10 different nanofibers in each sample. The addition of Amplex red does not have any significant effect on the nanofiber's diameter.

FIG. 4D is an image depicting (D) FTIR analysis was also performed on silk fibroin mats and Amplex red infused silk fibroin mats. The presence of amide region peaks in the range of 1600 cm⁻¹, 1500 cm⁻¹ and 1200 cm⁻¹ indicates the presence of random coils and beta sheets conformations of silk fibroin indicating the aqueous stability of nanofibers. The presence of other specific peaks in the region of 2900 cm⁻¹, 2800 cm⁻¹, 1000 cm⁻¹ and 950 cm⁻¹ indicates the infusion of Amplex red compound into the nanofibrous silk fibroin mats. (R) represents the random coils conformations of the silk fibroin while (B) represents the beta-sheets conformation bands of the silk fibroin

FIG. 5A is an image depicting Amplex red compound infused silk fibroin nanofibrous mats is being tested for the visual detection of H₂O₂. (A) represent control (silk fibroin mats) and Amplex infused silk fibroin mats (thick˜0.0043 mm thickness and thin one˜0.001 mm thickness) being tested. The thick and thin nanofibrous silk fibroin mats efficiently develop the pink color through the oxidation of Amplex red into Resorufin.

FIG. 5B is an image depicting Amplex red compound infused silk fibroin nanofibrous mats is being tested for the visual detection of H₂O₂. (B) represents the dimensions of electrospun silk fibroin mats used for further sensing measurement. These dimensions were 1×0.5 cm (length×height) for sensing measurements.

FIG. 5C is an image depicting Amplex red compound infused silk fibroin nanofibrous mats is being tested for the visual detection of H₂O₂. (C) Amplex infused silk fibroin mats treated with 2 different concentrations of H₂O₂ and HRP were kept at 100 folds apart in concentration. A control mat is shown at the top left corner for color development comparison. The mats efficiently develop the visible color on being treated with hydrogen peroxide.

FIG. 5D is an image depicting (D) Surface elemental analysis of control silk fibroin mats, Amplex infused silk fibroin mats, and Amplex infused silk fibroin mats that have undergone a color change through the oxidation of Amplex red component. Survey spectra (i) silk fibroin mats, (ii) Amplex infused silk fibroin mats: unoxidized, and (iii) Amplex infused silk fibroin mats: oxidized and C 1s spectra of respective silk fibroin mats with the deconvolution of the experimental spectra results in peaks corresponding to the binding energy of C—C/C═C, C—N, C—O and C═O. Fitted and actual spectra are shown.

FIG. 6A is an image depicting cellular biocompatibility analysis was performed for Amplex Red compound using the Human Skin keratinocyte (HaCat) cells through measuring the cellular viability by MTT assay. Cellular viability beyond 80% was considered biocompatible. Different concentration of Amplex Red compound was used and concentration up to 388 μM was found to be highly biocompatible when tested for 24 hours cellular biocompatibility test.

FIG. 6B is an image depicting cellular biocompatibility analysis was performed for Amplex Red compound using the Human Skin keratinocyte (HaCat) cells through measuring the cellular viability by MTT assay. Cellular viability beyond 80% was considered biocompatible. Different concentration of Amplex Red compound was used and concentration up to 388 μM was found to be highly biocompatible when tested for 48 hours cellular biocompatibility test.

FIG. 7A is an image depicting Control and Amplex infused silk fibroin mats treated with fixed concentrations of HRP and various concentrations of H₂O₂ ranging from 1 mM to 1 μM to find a limit of detection of the silk fibroin mats with quantification of the change of the color intensity done by ImageJ software. (A) Control and Amplex infused silk fibroin mats in the presence of a fixed HRP concentration of 25 μg/mL and various H₂O₂ concentrations at 30 minute, 24 hour, and 48 hour time point.

FIG. 7B is an image depicting Control and Amplex infused silk fibroin mats treated with fixed concentrations of HRP and various concentrations of H2O2 ranging from 1 mM to 1 μM to find a limit of detection of the silk fibroin mats with quantification of the change of the color intensity done by ImageJ software. (B) Control and Amplex infused silk mats in the presence of a fixed HRP concentration of 0.25 μg/mL and various H2O2 concentrations at 30 minute, 24 hour, and 48 hour time points.

FIG. 7C is an image depicting Control and Amplex infused silk fibroin mats treated with fixed concentrations of HRP and various concentrations of H₂O₂ ranging from 1 mM to 1 μM to find a limit of detection of the silk fibroin mats with quantification of the change of the color intensity done by ImageJ software. (C) Quantification of color intensity for the mats treated with 0.25 μg/mL HRP at the 24 hour time point.

FIG. 7D is an image depicting Control and Amplex infused silk fibroin mats treated with fixed concentrations of HRP and various concentrations of H₂O₂ ranging from 1 mM to 1 μM to find a limit of detection of the silk fibroin mats with quantification of the change of the color intensity done by ImageJ software. (D) Quantification of color intensity for the mats treated with 0.25 μg/mL HRP at the 48 hour time point.

FIG. 8 is an image depicting H₂O₇ color sensing with Amplex infused silk fibroin mats were performed using a diabetic (Db/Db) mouse model of wound healing. Control silk fibroin mats, H₂O₂ color sensing silk fibroin mats (thin˜0.001 mm thickness) and H₂O₂ color sensing silk fibroin mats (thick˜0.0043 mm thickness) i.e. Amplex infused silk fibroin mats were used. 8 mm dermal wounds were created onto the skin of diabetic mice with these mats were applied respectively as shown in the images. Upon observation it was noted that, within 24 h of time period the H₂O₂ color sensing silk fibroin mats white color changed to pink color due to the encountering of H2O: oxidative molecules and peroxidase exudating from the wound site, indicating the high concentration over 24 h time period

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that there are other embodiments by which the invention may be practiced. It is to be understood that other embodiments may be utilized and structural changes may be made without departing from the scope of the invention.

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

As used in the specification and claims, the singular form “a”, “an” and “the” include plural references unless the context clearly dictates otherwise.

As used herein, the term “comprising” is intended to mean that the products, systems and methods include the referenced components or steps, but not excluding others. “Consisting essentially of” when used to define products, systems and methods, shall mean excluding other components or steps of any essential significance. “Consisting of” shall mean excluding more than trace elements of other components or steps.

All numerical designations, including ranges, are approximations which are varied up or down by increments of 1.0 or 0.1, as appropriate. It is to be understood, even if it is not always explicitly stated that all numerical designations are preceded by the term “about”.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

The term “about” or “approximately” as used herein refers to being within an acceptable error range for the particular value as determined by one of ordinary skill in the art, which will depend in part on how the value is measured or determined. As used herein the term “about” refers to +10% of the numerical.

“Optional” or “optionally” means that the subsequently described event or circumstance may or may not occur, and that the description includes instances where said event or circumstance occurs and instances where it does not.

The term “fibroin” as used herein refers to silkworm fibroin. In some embodiments the silkworm fibroin is obtained from a solution containing dissolved silkworm silk. It is to be noted however, that other silks are contemplated for use in the invention including, but not limited to, insect silk protein; spider silk protein (e.g. obtained from Nephil clavipes); transgenic silks; genetically engineered silks, such as those obtained from bacteria, yeast, mammalian cells, transgenic animals, or transgenic plants; and variants thereof.

“Treatment” or “treating” as used herein refers to any of the alleviation, amelioration, elimination and/or stabilization of a symptom, as well as delay in progression of a symptom of a particular disorder. For example, “treatment” may include any one or more of the following: amelioration and/or elimination of one or more symptoms associated with infection or pain at the wound site, reduction of one or more symptoms of infection or pain at the wound site, stabilization of symptoms of infection or pain at the wound site, and delay in progression of one or more symptoms of infection or pain at the wound site.

“Infection” as used herein refers to the invasion of one or more microorganisms such as bacteria, viruses, fungi, yeast or parasites in the body of a patient in which they are not normally present.

The terms “administer” or “administering” as used herein are defined as the process by which the compositions of the present invention or other therapeutic agents are delivered to the patient for treatment or prevention purposes. Therapeutic agents can be delivered topically in dosage unit formulations containing conventional nontoxic pharmaceutically acceptable carriers, adjuvants, and vehicles as desired. Administration may occur once or multiple times. The electrospun nanofibrous silk fibroin mats may be applied prior to administration of the therapeutic agent as well as after administration of the therapeutic agent. “Applying” the mats to the wound site refers to substantially covering the wound site with the mat. “Substantially” as used herein, with respect to applying the mat to the wound site, refers to covering at least about 80% of the wound site with the mat. Preferably 100% of the wound is covered with the mat.

The term “therapeutic agent” as used herein refers to substance, component or agent that has measurable specified or selective physiological activity when administered to an individual in a therapeutically effective amount. Examples of active agents as used in the present invention include antimicrobials such as antibiotics, antivirals, antifungals, antiprotozoals, and antiparasitics; antioxidants; natural therapies such as honey; recombinant glucose oxidase; galvanic particles; hyperbaric oxygen therapy; recombinant PDGF; recombinant Galectin-1; topical application of ROS intermediates such as hydrogen peroxide, tetrachlorodecaoxide, and benzoyl peroxide; or combinations thereof.

A “therapeutically effective amount” as used herein is defined as concentrations or amounts of components which are sufficient to effect beneficial or desired clinical results, including, but not limited to, any one or more of treating symptoms of infection and preventing infection.

“Patient” is used to describe an animal, preferably a mammal, more preferably a human, to whom treatment is administered, including prophylactic treatment with the compositions of the present invention.

The term “ROS detectable reagent” as used herein refers to any reagent capable of detecting reactive oxygen species (ROS) that comprises a label that is observable visually or through the use of a machine designed for the detection of the label. Any ROS detection probe is contemplated under this invention provided the probe is stable under the fabrication conditions described herein. ROS comprise chemically reactive oxygen radicals as well as non-radical derivatives of oxygen such as hydrogen peroxide. Examples of ROS include superoxide (O⁻₂), hydrogen peroxide (H₂O₂), hydroxyl radical (.OH), singlet oxygen (¹O₂), and peroxynitrite (ONOO⁻). Exemplary ROS detectable agents include, but are not limited to, Amplex red and AmplexUltra red as well as any other probe that is stable under the fabrication conditions.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular ROS reactive electrospun nanofibrous mat is disclosed and discussed and a number of modifications that can be made to a number of molecules including the ROS reactive electrospun nanofibrous mat are discussed, specifically contemplated is each and every combination and permutation of ROS reactive electrospun nanofibrous mat and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood and herein contemplated that the electrospun nanofibrous mat does not have to be a fibrous sheet with a single use of detecting ROS levels, but can be incorporated into items used to treat the wound including, but not limited to, adhesive bandages, wound dressings, surgical drapes, and sutures. Thus, in one aspect, disclosed herein are electrospun nanofibrous mats, wherein the mat is a component of an adhesive bandage, wound dressing, surgical drape, or suture.

As used herein, the “detectable reagent” is any reagent that comprises a label that is observable visually or through the use of a machine designed for the detection of the label (such as, for example, by spectrophotometric or fluorometric means with a UV spectrometer, IR spectrometer (including, but not limited to FTIR), fluorescence spectrometer, visual spectrometer, X-Ray spectrometer, or the like). A label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorimetric substrates (e.g., horseradish peroxidase). In some embodiments, fluorescent dyes are used in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody. In one aspect, the label of the detectable reagent can comprise Amplex Red.

Fluorophores are compounds or molecules that luminesce. Typically, fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2, 7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); AB Q; Acid Fuchsin; Acridine Orange; Acridine Red; Acridine Yellow; Acriflavin; Acriflavin Feulgen SITSA; Aequorin (Photoprotein); AFPs—AutoFluorescent Protein-(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATIO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; BodipyS00/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-I Ca2+ Dye; Calcium Green-2 Ca2+; Calcium Green-SN Ca2+; Calcium Green-IO C18 Ca2+; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hep; Coelenterazine ip; Coelenterazine n; Coelenterazine O; Coumarin Phalloidin; C-phycocyanine; CPM I Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (Di1C18(3)); I Dinitrophenol; DiO (DiOC18(3)); DiR; DiR (Di1C18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Pura Red™ (high pH); Pura Red™/Fluo-3; Fura-2; Fura-2/BCECF; Genacryl Brilliant Red B; Genacryl Brilliant Yellow 1OGF; Genacryl Pink 3G; Genacryl Yellow SGF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Pura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-I; TOTO-3; TriColor (PE-Cy5); TR1TC TetramethylRodaminelsoThioCyanate; True Blue; Tru Red; Ultralite; Uranine B; Uvitex SFC; wt GFP; WW 781; X-Rhodamine; XR1TC; Xylene Orange; Y66F; Y66H; Y66W; Yellow GFP; YFP; YO-PRO-1; YO-PRO 3; YOYO-1;YOYO-3; Sybr Green; Thiazole orange (interchelating dyes); semiconductor nanoparticles such as quantum dots; or caged fluorophore (which can be activated with light or other electromagnetic energy source), or a combination thereof.

Wound healing is of major concern in day to day clinical care and is constantly being explored for early restoration and enhanced recovery. While the etiology of wound healing is multifactorial, high inflammation and increased oxidative stress which results in chronic inflammation, endothelial dysfunction and collagen degradation, delay the overall healing process. Thus, visual sensing of the oxidative stress would be highly informative in the successful implementation of wound healing therapies based on specific requirements. The visual identification and measurement of oxidative stress directly into these wounds indicates the level and stage of the effective healing process. Identifying the level of oxidative stress assists in determining an effective overall therapeutic approach to accelerate wound healing.

To solve the problem of ROS detection at the wound site, the inventors have developed a novel mat and corresponding method that gives a visible change in color that can quickly provide a measurement of ROS levels in real time in the wounds. This method detects the level of oxidative stress in a wound site and facilitates the decisions and drugs to be introduced and further interventions required. Exemplary therapeutic strategies that can be utilized in response to ROS level in a wound as detected by the novel nanofibrous mat described herein include, but are not limited to, administration of antioxidants; natural therapies such as honey; antibiotics; recombinant glucose oxidase; galvanic particles; hyperbaric oxygen therapy; recombinant PDGF; recombinant Galectin-1; topical application of ROS intermediates such as hydrogen peroxide, tetrachlorodecaoxide, and benzoyl peroxide; or combinations thereof. In an embodiment, the color change an initial measurement of ROS level in the wound can be compared to color change in a measurement taken after therapy has been administered to the wound site to determine the effectiveness of the therapy on the wound site. In an Example herein, a ROS reactive detectable reagent (e.g., Amplex Red) was infused into an electrospun nanofibrous mat (e.g., a silk mat).

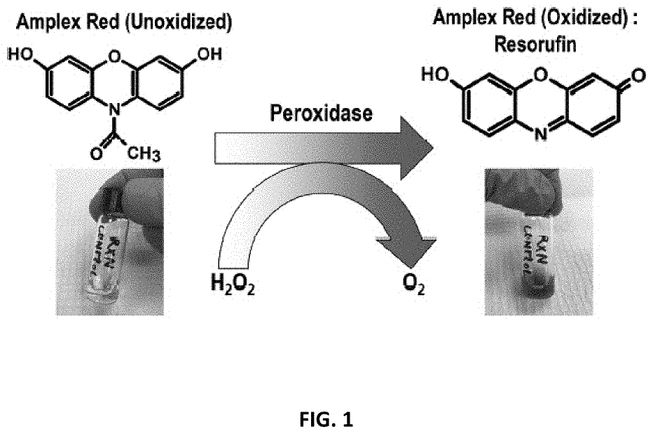

Amplex Red is a highly sensitive and stable fluorogenic probe used to detect and quantify H₂O₂. The detection of H₂O₂ relies on the oxidation of Amplex Red into resorufin in the presence of horseradish peroxidase (HRP) as shown in FIG. 1. Amplex Red is a colorless and non-fluorescent compound, but reacts in 1:1 ratio with H₂O₂ to produce the highly fluorescent resorufin and detect as little as 10 picomoles of H₂O₂ in a 100 μL volume. Reports have shown that when used in an assay, there is a visible change in color from clear solution to pink/purple which is ideal in laboratory settings to ensure the color change is due to H₂O₂ presence. (W. M. Aumiller Jr., B. W. Davis, N. Hashemian, C. Maranas, A. Armaou and C. D. Keating, J. Phys. Chem. B, 2014, 118, 2506-2517; B. Zhao, F. A. Summers and R. P. Mason, Free Radicals Biol. Med., 2012, 53, 1080-1087). With this knowledge regarding Amplex Red, as well as its common use in vitro, the inventors chose Amplex Red as the dye used with the electrospun nanofibrous mats.

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective, and for a period of time sufficient, to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

In the Example provided herein, the material used for electrospinning with Amplex Red was silk obtained from the Bombyx mori silk cocoons. Silk fibroin has been used in several biomedical applications including sutures in surgeries, linen, and hydrogels for tissue engineering. (C. Holland, K. Numata, J. Rnjak-Kovacina and F. P. Seib, Adv. Healthcare Mater., 2019, 8, 1800465). Biomedical applications of silk fibroin are enabled due to characteristics of the material that make it suitable for these purposes, including robustness easy chemical modification of surface properties, good biocompatibility, and slow degradation. (D. N. Rockwood, R. C. Freda, T. Yücel, X. Wang, M. L. Lovett and D. L. Kaplan, Nat. Protoc., 2011, 6, 1612).

Silk fibroin nanofibrous mats were fabricated using electrospinning technology leading to the deposition of evenly distributed micro to nano diameter ranged silk nanofibers into mats. This process is highly useful in controlling the surface morphology and other material properties and also offers the flexibility to incorporate substances of interest such as organic dyes like Amplex Red in this case. (S. Aziz, M. Sabzi, A. Fattahi and E. Arkan, J. Polym. Res., 2017, 24, 140). Thus, the inventors found that integrating Amplex red in silk fibroin solution and fabricating electrospun silk fibroin nanofibrous mats provided a method of visible and rapid detection of ROS level in the external wound environment to provide insight into the overall wound healing process.

Also disclosed herein are methods of detecting reactive oxygen species (ROS) levels at a wound site comprising applying an electrospun nanofibrous mat of any preceding aspect to the wound site and detecting a change in color of the electrospun nanofibrous mat; wherein the presence of a change in color indicates the presence of ROS. ROS color changes can be detected by any means known in the art including, but not limited to infrared spectroscopy, ultraviolet spectroscopy, fluorescence spectroscopy, visible spectroscopy, and/or X-ray photoelectron spectroscopy (XPS).

In one aspect, disclosed herein are methods of detecting ROS levels at a wound site of any preceding aspect, further comprising comparing the degree of color change in the electrospun nanofibrous mat relative to a control to measure the amount of ROS at the wound site.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Nanofibrous Silk Mat for Visible Sensing of Oxidative Stress in Cutaneous Wounds

The inventors fabricated a flexible electrospun silk fibroin nanofibrous mat which is highly sensitive to oxidative stress environments and can visibly detect the presence of hydrogen peroxide with a change in color, especially when applied on cutaneous wounds. This hydrogen peroxide sensing platform can be used to determine efficient therapies based on the wound healing stage.

Electrospinning of Silk Fibroin Nanofibrous Mats and SEM, FTIR Characterization

In this direction, a highly clear and semi-viscous silk fibroin solution was prepared from raw silk Bombyx mori cocoons employing the previously established protocol for fabricating electrospun silk fibroin mats described in Rockwood 2011 and Saraf 2019, herein incorporated into this disclosure in their entirety. (.D. N. Rockwood, R. C. Freda, T. Yücel, X. Wang, M. L. Lovett and D. L. Kaplan, Nat. Protoc., 2011, 6, 1612; N. Saraf, S. Barkam, M. Peppier, A. Metke, A. Vazquez-Guardado, S. Singh, C. Emile, A. Bico, C. Rodas and S. Seal, Sens. Actuators, B, 2019, 283, 724-730). This silk preparation process was used because the final silk fibroin solution is in an aqueous state and provides the flexibility of doping with any external materials or compounds. In an embodiment, the inventors doped the silk fibroin solution with Amplex red prior to electrospinning Using the drying technique, the concentration of silk fibroin solution obtained was 7%. Concentration of silk in the silk fibroin solution can range from about 5% to about 15%. Varying the silk concentration in the silk fibroin solution changes the nanofiber diameter in electrospinning Thus, the size of the nanofibers in the mat can be tuned as needed according to the silk concentration.

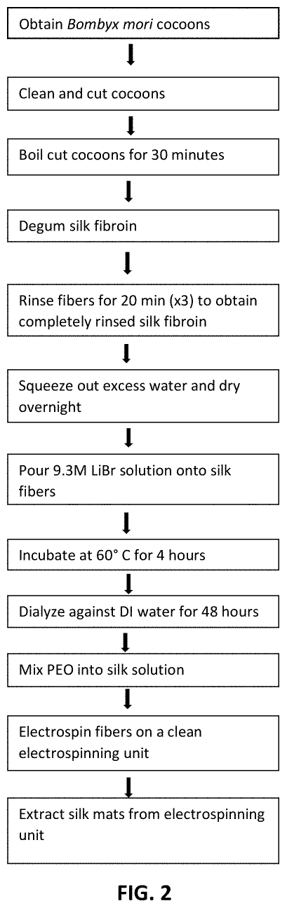

The protocol used for fabrication of the nanofibrous mats is shown in FIG. 2. Generally, as shown in the figure, Bombyx mori cocoons were cleaned and cut into smaller pieces after which the cocoons were boiled for about 30 minutes. The silk fibroin is degummed and the fibers rinsed for about 20 minutes, three times, to obtain completely rinsed silk fibroin. Excess water was removed and the fibers were dried overnight. A 9.3M solution of LiBr was poured onto the silk fibers and incubated at 60° for about 4 hours. The fibers were then dialyzed against deionized water for 48 hours. An amount of PEO was mixed into the silk solution to form a silk solution. The electropsinning unit was cleaned and the silk solution was used to electrospin the fibers using the electrospinning unit by first drawing the silk solution into a 5 mL syringe attached to 23G needle and mounted to the syringe pump unit. The electrospinning unit was grounded with positive voltage lead connected to the solution containing syringe needle and the ground lead to the collector surface. The flow rate was adjusted to 1 mL/hr and the current was set to 2 A and electric potential to 20 kV. The distance between the syringe needle and the collector drum was set to 10 cm apart. Electrospinning of silk mat was performed until a visible mat of suitable thickness was collected onto the collector unit after which the silk mats were extracted from the electrospinning unit.

This general protocol was also used for fabrication of the Amplex red infused nanofibrous silk fibroin mats with the Amplex red-silk solution substituted for the silk solution of the control in the electrospinning unit. A stock of Amplex red solution was prepared in DMSO at a concentration of 5 mg/mL and mixed thoroughly. From this stock preparation, 1 ml of Amplex red solution was added to the 45 ml of silk solution (silk concentration=54.4 mg/mL) under mild stirring condition in increment of 200 μl every 5 minutes. This Amplex red-silk fibroin solution was stored under 4° C. and used for electrospinning of nanofibrous silk fibroin mats. This Amplex red-silk fibroin solution was drawn in a 5 mL syringe attached to 23G needle and mounted to the syringe pump unit. The electrospinning unit was grounded with positive voltage lead connected to the solution containing syringe needle and the ground lead to the collector surface. The flow rate was adjusted to 1 mL/hr and the current was set to 2 A and electric potential to 20 kV. The distance between the syringe needle and the collector drum was set to 10 cm apart. Electrospinning of silk mat was performed until a visible mat of suitable thickness was collected onto the collector unit

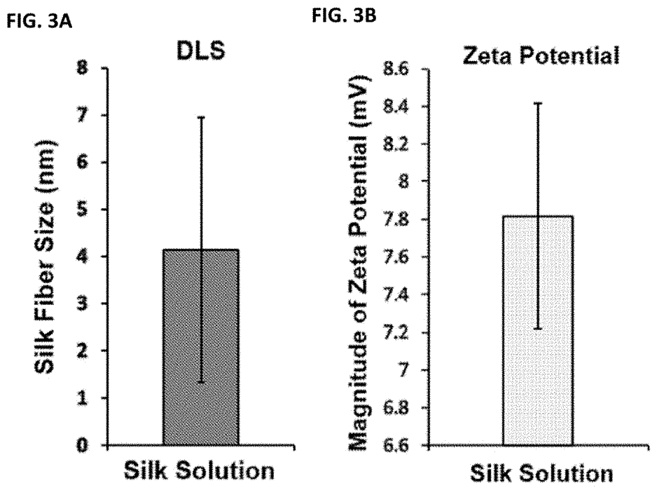

These silk fibroin solutions were characterized for nanofiber diameter and surface charge potential. Nanofibrous silk diameter of silk solution was estimated using the dynamic light scattering (DLS) measurement and was observed to have an average nanofiber size of 4.13±2.8 nm (FIG. 3A). These silk nanofibers were highly homogenous in measurement. Zeta potential analysis performed onto these nanofibrous silk fibroin solutions was determined to be 7.82±0.6 mV (FIG. 3B). Values lower than ±5 mV are generally considered as unstable in aqueous solution and also these solution tends to aggregate more easily and rapidly. Overall, the nanofiber size and surface charge analysis indicated that the prepared nanofiber silk solution to be highly suitable for electrospinning nanofibrous silk mats.

These silk fibroin solutions were processed for electrospinning by mixing with 5 mL of 5% polyethylene oxide (PEO) before electrospinning. It has been reported that PEO addition to silk fibroin solution induces ample surface tension and sufficient viscosity so that a continuous fibroin jetting can be maintained, a requirement for the efficient electrospinning of nanofibrous mats. (A. J. Meinel, K. E. Kubow, E. Klotzsch, M. Garcia-Fuentes, M. L. Smith, V. Vogel, H. P. Merkle and L. Meinel, Biomaterials, 2009, 30, 3058-3067; C. Li, C. Vepari, H.-J. Jin, H. J. Kim and D. L. Kaplan, Biomaterials, 2006, 27, 3115-3124). The silk fibroin nanofibrous mats were electrospun from the silk fibroin solution blended with polyethylene oxide (PEO), which is also a biocompatible polymer. (H.-J. Jin, S. V. Fridrikh, G. C. Rutledge and D. L. Kaplan, Biomacromolecules, 2002, 3, 1233-1239; L. Huang, K. Nagapudi, R. P. Apkarian and E. L. Chaikof, J. Biomater. Sci., Polym. Ed., 2001, 12, 979-993; L. G. Griffith, Acta Mater., 2000, 48, 263-277). This also minimizes the potential toxicity which may arise from the use of any organic solvent and may later affect the applicability of these mats in vitro and in vivo. While PEO was used in the examples, other biocompatible polymers are contemplated for use in the invention including, but not limited to, polyethylene glycol; collagen; fibronectin; keratin; polyaspartic acid; polylysin; alginate; chitosan; chitin; hyaluronic acid; or variants thereof.

Silk fibroin electrospinning was performed and silk fibroin nanofibrous mats (Control) were obtained on the metal collector unit. Similarly, both Amplex infused silk fibroin nanofibrous mats were synthesized through electrospinning technique. These electrospun control silk fibroin mats and Amplex infused silk fibroin mats were processed further in small sizes for SEM, FTIR and biochemical testing. The electrospinning technique was applied because it creates numerous beneficial features in these mats including high porosity created by the electrospun nanofibers which helps in the absorption of the exudates in the wound and efficient gas exchange which supports the wound cells migration and promote cellular proliferation.

Also, the Amplex red has been exclusively used for the detection of H₂O₂ in solutions with a detection limit as low as 10 picomolar. Amplex red is an ultrasensitive compound used in combination with HRP for the quantitative determination of hydrogen peroxide as a marker of oxidative stress. Combination of Amplex red and HRP has been used to estimate the H₂O₂ generation in native and recombinant microsomal preparations of cytochrome P450. (V. Mishin, J. P. Gray, D. E. Heck, D. L. Laskin and J. D. Laskin, Free Radicals Biol. Med., 2010, 48, 1485-1491). Similarly, Amplex red has been used for estimating oxidative stress through H₂O₂ in polymeric hydrogel spheres as well in mammalian cell culture system of human respiratory epithelial A549 cells. (S.-H. Kim, B. Kim, V. K. Yadavalli and M. V. Pishko, Anal. Chem., 2005, 77, 6828-6833; V. W. Wong, K. C. Rustad, J. P. Glotzbach, M. Sorkin, M. Inayathullah, M. R. Major, M. T. Longaker, J. Rajadas and G. C. Gurtner, Macromol. Biosci., 2011, 11, 1458-1466; L. S. Gloyne, G. D. Grant, A. V. Perkins, K. L. Powell, C. M. McDermott, P. V. Johnson, G. J. Anderson, M. Kiefel and S. Anoopkumar-Dukie, Toxicol. in Vitro, 2011, 25, 1353-1358). The infusion of silk fibroin solutions with different compounds and dyes and electrospun into nanofibrous silk fibroin mats have also been reported. It has been reported that applicable dyes or drug agents can be successfully infused into the silk fibroin nanofibers during the electrospinning process including FITC-albumin and riboflavin. (Y. D. Shanskii, N. Sergeeva, I. Sviridova, M. Kirakozov, V. Kirsanova, S. Akhmedova, A. Antokhin and V. Chissov, Bull. Exp. Biol. Med., 2013, 156, 146-151; K. Min, S. Kim, C. G. Kim and S. Kim, Sci. Rep., 2017, 7, 5448). Infusion of these molecules into electrospun nanofibers indicates that successful incorporation of Amplex red into the silk fibroin nanofiber can be achieved, which was further confirmed using the physiochemical characterization of the synthesized mats.

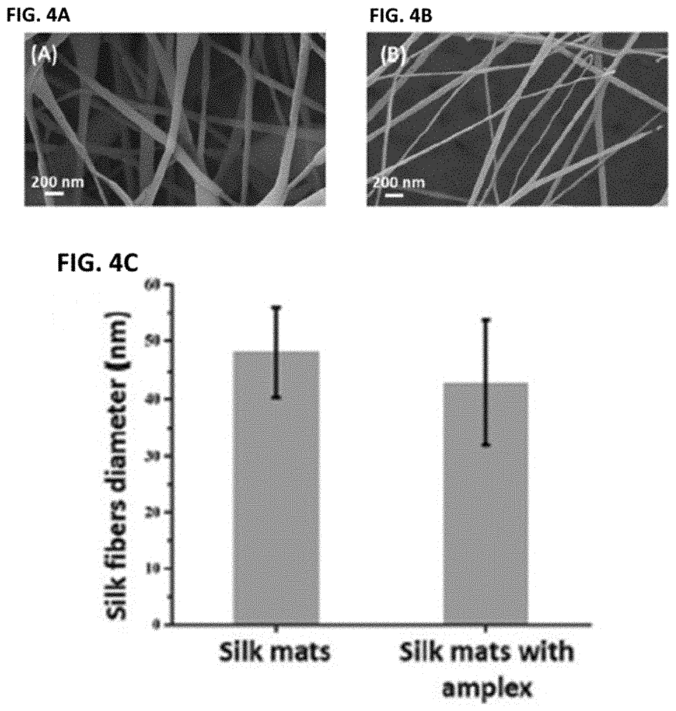

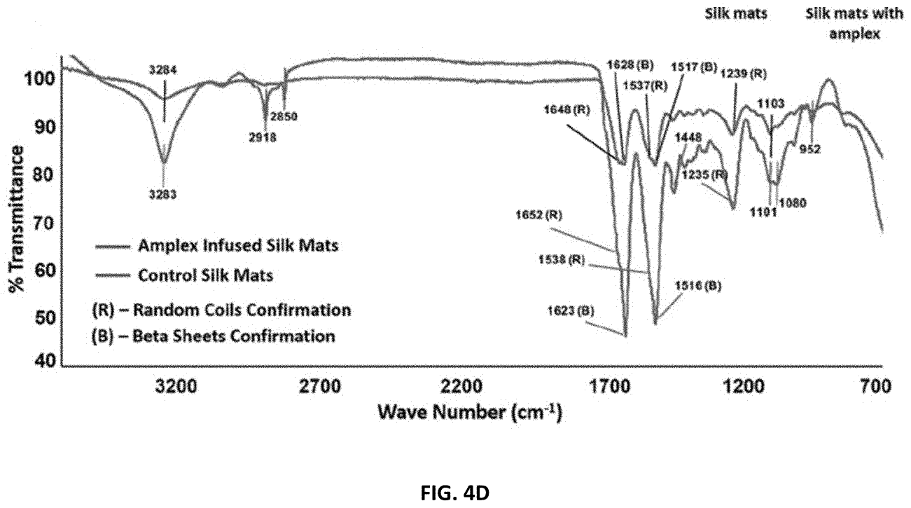

Electrospinning is a versatile technique allowing the formation of scaffolds which is ultra-structurally composed of highly porous micro/nanofibers arranged in uniform fashion depending on the electrospinning unit collector setup. (J. Doshi and D. H. Reneker, J. Electrost., 1995, 35, 151-160; S. V. Fridrikh, H. Y. Jian, M. P. Brenner and G. C. Rutledge, Phys. Rev. Lett., 2003, 90, 144502). Here, the electrospun nanofibrous silk fibroin mats and the Amplex-red infused nanofibrous silk fibroin mats were subjected to physiochemical and biochemical characterization. SEM was used to image the nanofibrous ultrastructure and identify if the infusion of Amplex red had any effect on the nanofibrous diameter during its electrospinning process and ultimately silk fibroin mats formation. Fine silk nanofibrous images were observed at a scale bar of 200 nm with the average nanofiber diameter around 50 nm. Similar nanofiber ultrastructure was also observed in both control and Amplex infused silk fibroin nanofibrous mats. (FIGS. 4A-C)

Both the control silk fibroin mats and Amplex red infused silk fibroin mats have similar nanofibrous ultrastructure indicating the lack of any effect on of Amplex red infusion before electrospinning. The nanofiber diameter of silk fibroin depends upon the route of materials synthesis, solvent types and ultimately electrospinning parameters. Reports indicated that nanofiber diameter can vary from 100 nm to maximum 1000 nm using the aqueous-based electrospinning of silk fibroin mats. The aqueous-based silk fibroin solution and nanofiber diameter obtained by the inventors was less than that reported earlier. (X. Zhang, M. R. Reagan and D. L. Kaplan, Adv. Drug Delivery Rev., 2009, 61, 988-1006; H. Wang, H. Shao and X. Hu, J. Appl. Polym. Sci., 2006, 101, 961-968).

It is also important to identify the chemical nature of the silk fibroin mats. To determine chemical nature, FTIR analysis was performed on both the control silk fibroin nanofibrous mats and the Amplex red infused silk fibroin nanofibrous mats (FIG. 4D). The FTIR spectra obtained for both of these silk fibroin mats were compared and analyzed. The 1103 cm⁻¹ band, likely caused by the C-C stretching of tyrosine aromatic rings, tryptophan or phenolic compounds, also appeared in previous studies on Bombyx mori silk characterization, and it appeared in the FTIR spectra of both of the control silk fibroin mats and Amplex infused silk fibroin mats. (M. Boulet-Audet, F. Vollrath and C. Holland, J. Exp. Biol., 2015, 218, 3138-3149). Amide regions peaks (Amide I, II, III) in the zone 1600s, 1500s and 1200s were highly prominent in both types of silk fibroin nanofibrous mats, indicating the presence of random coils and beta-sheet conformation. The electrospun silk fibroin nanofibrous control mats were composed of both the Silk-I (random coils) and Silk-II 03-sheet conformation) of silk fibroin. The FTIR data of control silk fibroin mats (FIG. 4D) indicates the characteristic band peaks for these random coils conformation (1648 cm⁻¹, 1537 cm⁻¹, 1239 cm¹) and β-sheet conformation (1628 cm⁻¹, 1517 cm¹). (D. J. Belton, R. Plowright, D. L. Kaplan and C. C. Perry, Acta Biomater., 2018, 73, 355-364; X. Hu, D. Kaplan and P. Cebe, Macromolecules, 2006, 39, 6161-6170). Reports have indicated that despite an intense band at 1650 cm¹ of Silk-I/random coils conformation, there is subtle amount of Silk-II/beta-sheet conformation present with less intense bands at 1622 cm¹. Thus, it's expected for electrospun silk fibroin mats to show preferentially random coil conformations but in coexistence with a minor proportion of beta-sheet structures. (D. J. Belton, R. Plowright, D. L. Kaplan and C. C. Perry, Acta Biomater., 2018, 73, 355-364; T. C. D. Fernandes, H. M. R. Rodrigues, F. A. A.

Paz, J. F. M. Sousa, A. J. M. Valente, M. M. Silva, V. de Zea Bermudez and R. F. P. Pereira, J. Electrochem. Soc., 2020, 167, 070551). Also, the FTIR peaks of Amplex infused silk fibroin mats were in close coincidence with control silk fibroin mats Amplex infused silk fibroin mats contained high-intensity peaks in the 1623 cm⁻¹, 1516 cm¹ region corresponding to beta sheets structure and this is due to the interaction between DMSO present into Amplex red solution and silk fibroin when added for electrospinning (T. C. D. Fernandes, H. M. R. Rodrigues, F. A. A. Paz, J. F. M. Sousa, A. J. M. Valente, M. M. Silva, V. de Zea Bermudez and R. F. P. Pereira, J. Electrochem. Soc., 2020, 167, 070551). FTIR analysis also indicates that the control silk fibroin samples band were minor at 1628 cm¹ and 1517 cm¹, while the Amplex containing silk fibroin sample has a sharper band at 1623 cm¹ and 1516 cm¹, indicating the random coil transformation into β-sheet structures by the DMSO used for solubilizing Amplex compound for electrospinning the mats. Additional FTIR peaks appeared into the Amplex infused silk fibroin mats which may be due to the presence of the Amplex compound within the silk fibroin nanofibrous mats (FIG. 4D). The FTIR peaks at 1400 cm⁻¹ indicate the OH alcohol group bending, 1080 cm¹ indicate the C—O alcohol stretch while 953 cm¹ indicate the CvC stretch, specific functional groups associated with the Amplex red compound. The presence of these functional groups into the Amplex infused nanofibrous silk fibroin mats indicate the high infusion rate and retention of the complete chemical moiety of the Amplex compound even after the electrospinning procedure. Amplex retention is further confirmed through the biochemical testing which induces the visible color change immediately upon encountering the H₂O₂ and peroxidases, indicating the robustness of the chemical compound even through passing the high potential differences during the electrospinning process.

In Vitro Testing and XPS Analysis of Control and Amplex Infused Silk Fibroin Nanofibrous Mats

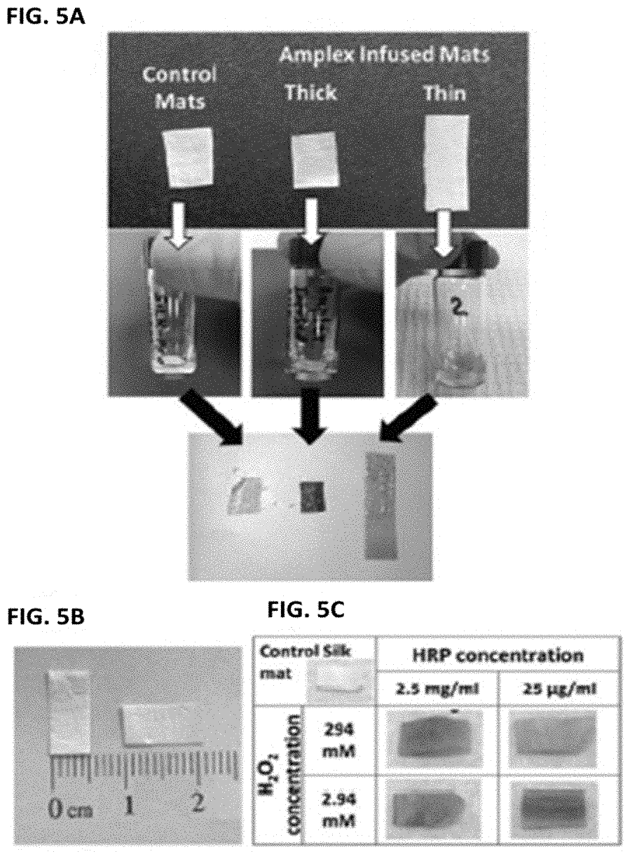

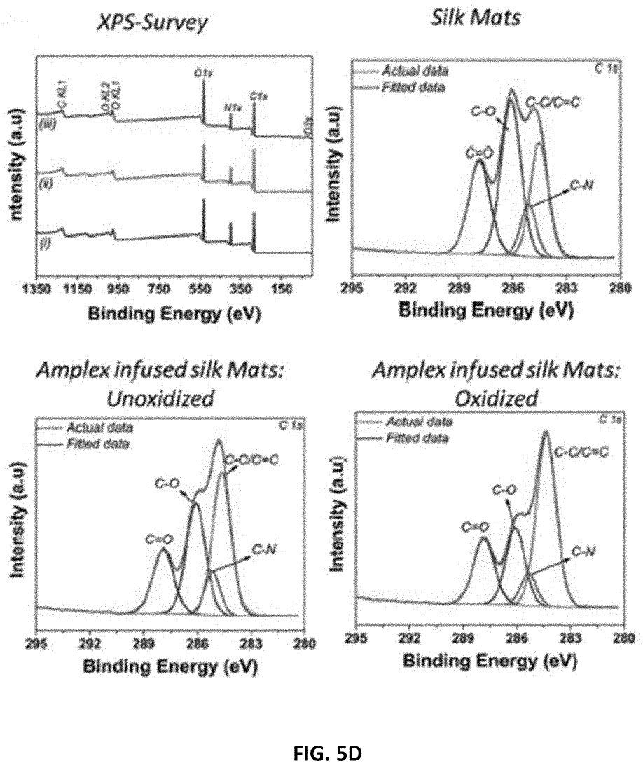

The chemical integrity of Amplex red infused silk fibroin mats was further tested with a visible change in color for sensing H₂O₂ moiety in solution. Both control silk fibroin mats and Amplex infused silk fibroin mats were tested using HRP and H₂O₂ for visible change in color from a transparent solution into pink/purple color. The Amplex red infused silk fibroin mats were found to oxidize and immediately produce a visible pink color upon interaction with HRP and H₂O₂ compounds (FIG. 5A). It has been reported that Amplex red limit of detection for H₂O₂ is up to 10 picomoles under specified conditions, which is much lower than the normal physiological H₂O₂ concentration with a cellular system

Amplex red oxidation into resorufin indicated its successful infusion within the silk fibroin ultrastructure and its robust chemical integrity which remains uncompromised during the electrospinning procedure. Amplex red withheld its functional chemical integrity through higher electric potential difference and from aqueous to solid phase. Further, these silk fibroin mats were cut into 1.0×0.5 cm dimensions and were tested with HRP and H₂O₂ at 2 different concentration 100-fold apart for the color development and sensitivity. Concentration of HRP was 2.5 mg/mL and 25 μg/mL while H₂O₂ concentration was 294 mM and 2.94 mM (FIGS. 5B and C). It was found that visual color development took place at both the concentration, indicating a highly robust nature of the Amplex infused silk fibroin mats and a wide range of sensitivity for H₂O₂ sensing. This analysis also directed us to proceed to lower H₂O₂ concentration for detection. To examine the detailed surface elemental composition, XPS was carried out on the control silk fibroin mats, Amplex infused silk fibroin mats, before and after the oxidation. The corresponding spectral lines are shown in FIG. 5D. The full survey spectral envelops of all silk fibroin mats contains C, O, N as the primary elements with different concentration of atomic %. (J. Shao, J. Liu, J. Zheng and C. M. Carr, Polym. Int., 2002, 51, 1479-1483). The relative concentration of C, O, N in the silk fibroin derivatives, quantified from the equivalent photoelectron peak area are presented in Table 1. The incremental atomic % of carbon (4.14%) from control silk fibroin mats to Amplex infused silk mats indicate that the Amplex red is successfully incorporated within the silk mat. Furthermore, atomic % of oxygen increased from unoxidized Amplex infused silk fibroin mats (20.27%) to oxidized silk fibroin mats (24.98%) is the clear evidence of oxidation reaction occurred within the silk fibroin mats.

To understand the associated local environmental modifications around carbon, the high-resolution C is envelope was deconvoluted and fitted into four peaks, namely, C—C/CvC. C—N, C—O, and CvO, centered at 284.5±0.2 eV, 285.14±0.2 eV, 286.12±0.2 eV, and 287.88±0.1 eV, respectively, as shown in FIG. 5D. (Q. Wang, R. Yanzhang, Y. Wu, H. Zhu, J. Zhang, M. Du, M. Zhang, L. Wang, X. Zhang and X. Liang, RSC Adv., 2016, 6, 34219-34224). CvO attributes the carbon on the peptide backbone groups associated with β-structures, while C—C/CvC reflects the aliphatic carbons of the amino acid pendant groups. The integrated peak area ratios (IPARi=Aoi/ΣAo) for C-C/CvC, C-N, C-O, CvO are presented in Table 1. The changes in the peak area ratio of the different carbon species indicate that reaction occurred within the unoxidized Amplex infused silk fibroin mats (before introduction to H₂O₂) and oxidized Amplex infused silk fibroin mats (after introduction to H₂O₂ and color development). Similarly, the high-resolution XPS 0 is spectra show the associated local environment modification around oxygen and nitrogen. Therefore, it is concluded that Amplex is infused into the silk fibroin mats and that a reaction does occur with the oxidation of Amplex into resorufin within the silk fibroin mats, indicated with the visible color change.

TABLE 1
Composition (atomic %) of silk fibroin mats, Amplex infused silk
fibroin mats: oxidized and unoxidized, as well as the peak area % of each
carbon components identified from C 1s spectra of respective silk fibroin mats

| | Atomic % | | | C1s~peak area % | | | |
|---|---|---|---|---|---|---|---|
| Sample | C1s | N1s | 01s | C~C/C═C | C~N | C~0 | C═0 |
| Silk fibroin mats | 62.60 | 15.81 | 21.58 | 25.9 | 10.4 | 38.7 | 25.0 |
| Amplex infused silk fibroin mats: unoxidized | 66.74 | 12.99 | 20.27 | 39.1 | 9.9 | 31.7 | 19.3 |
| Amplex infused silk fibroin mats: oxidized | 62.25 | 12.77 | 24.98 | 50.5 | 8.5 | 21.8 | 19.2 |

Cellular Biocompatibility for Amplex Compound and Amplex Infused Nanofibrous Silk Fibroin Mats LOD Analysis

Silk fibroin is known to be an excellent biomaterial, highly biocompatible and has been in use for generations. It has found its applications in sutures and other biomedical applications. The unique features include the high mechanical strength, biocompatible nature and varied ability of the silk protein to change its structural and morphological features. Silk fibroin is applied across various grafts including skin, bone and vascular tissues. (H.-J. Jin, S. V. Fridrikh, G. C. Rutledge and D. L. Kaplan, Biomacromolecules, 2002, 3, 1233-1239; I. Dal Pra, A. Chiarini, A. Boschi, G. Freddi and U. Armato, Int. J. Mol. Med., 2006, 18, 241-247; K.-H. Kim, L. Jeong, H.-N. Park, S.-Y. Shin, W.-H. Park, S.-C. Lee, T.-I. Kim, Y.-J. Park, Y.-J. Seol and Y.-M. Lee, J. Biotechnol., 2005, 120, 327-339).

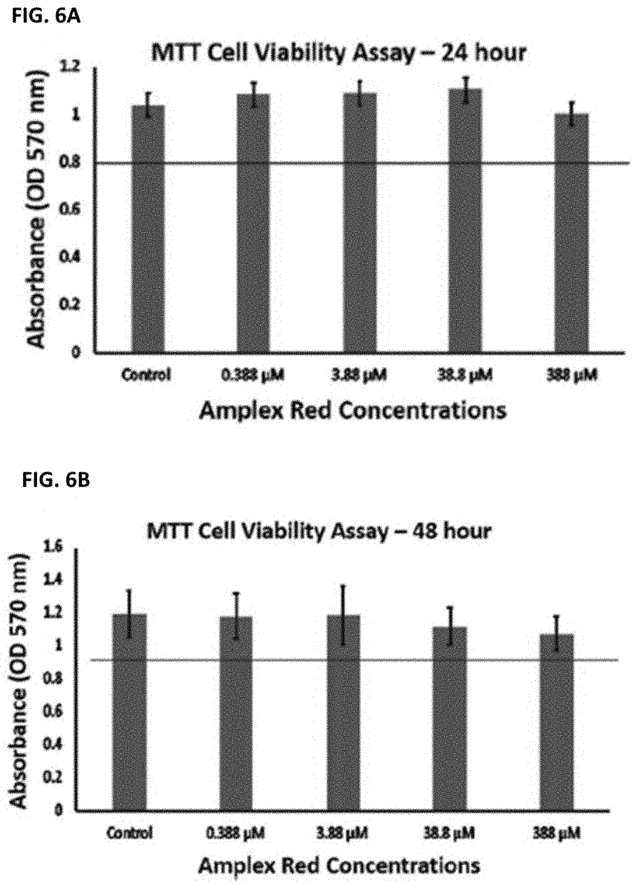

The silk fibroin mats developed here used Amplex red compound for H₂O₂ sensing. It is important to identify the cellular biocompatibility of the Amplex red compound and the Amplex red infused silk fibroin mats. Cellular biocompatibility was tested using human skin keratinocyte (HaCat) cells employing the standard MTT assay. It was observed that Amplex red was completely non-toxic to the cells. The 24 hours and 48 hours MTT data produced the cellular viability beyond 80% which indicates its nontoxic nature at the highest concentration of 388 μM, indicating its potential application into the cell-based detection system (FIG. 6A-B).

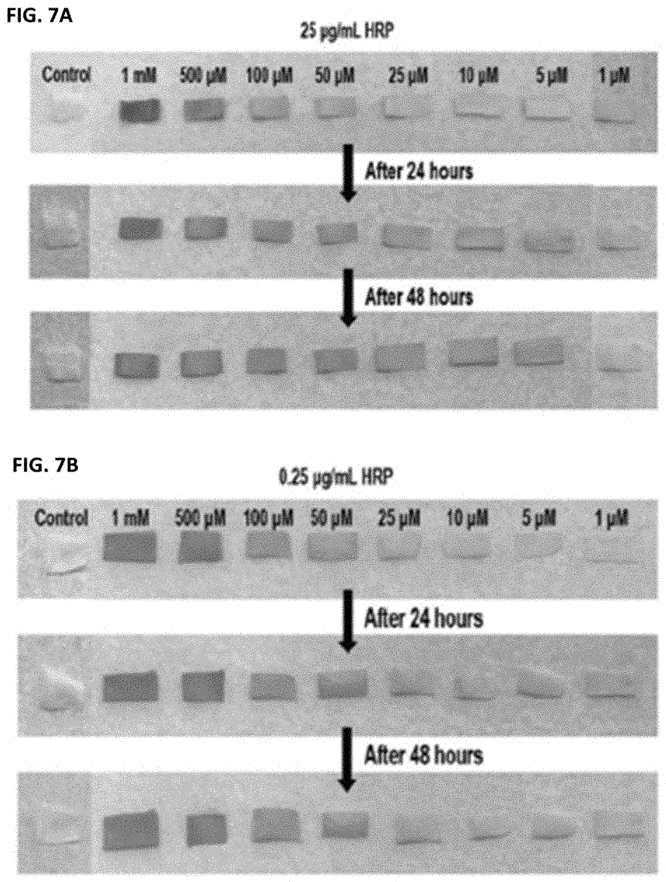

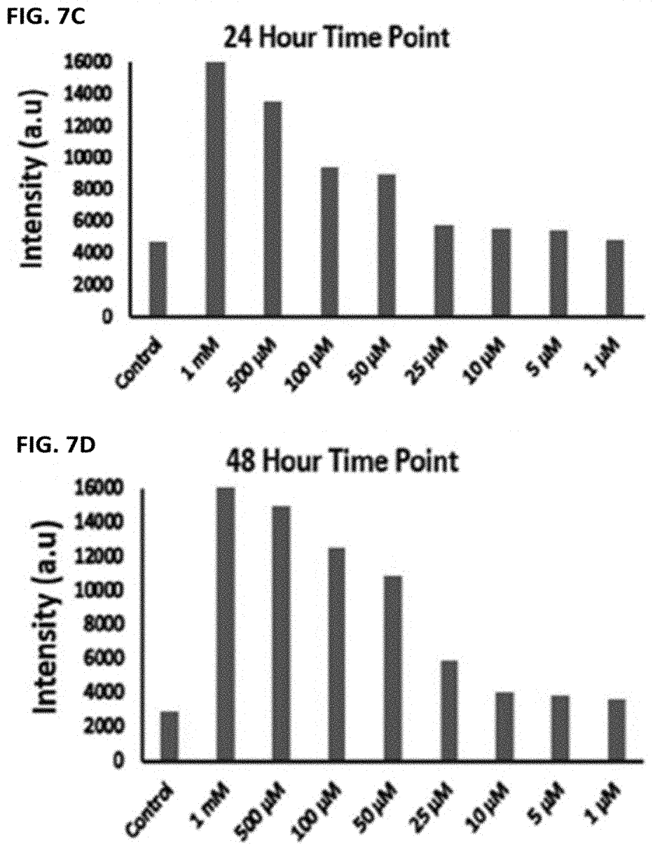

In a previous experiment, HRP and H₂O₂ levels were tested across 2 different concentrations that were 100 folds apart. The lower level of HRP was 25 μg/mL while the level of H₂O₂ was 29.4 mM. The visible color development at this concentration of HRP and H₂O₂ were immediate. The HRP concentration of 25 μg/ml produces a satisfactory and immediate visible color change in the presence of H₂O₂. Further limit of detection for H₂O₂ was analyzed keeping the concentration of HRP constant at 25 μg/mL and thus varying the level of H₂O₂ concentration from a higher level of 1 mM to a lower level of 1 μM. A total of 8 different concentrations were tested for visible change in color with respect to control mats under similar temperature and humidity. The Amplex red infused nanofibrous silk fibroin mats developed the visible color change immediately upon H₂O₂ interaction. Although the visible color change was observed immediately, the analysis was done at 2 different time points of 24 hours and 48 hours. This is done to observe the degree of color change over time duration within the presence of HRP and H₂O₂. Post-incubation the inventors observed that the initial developed visible color intensity did not change after 48 hours. Also, the Amplex red infused silk fibroin mats at the lowest concentration of 1 μM H₂O₂ developed color, indicating the limit of detection to be much lower (FIG. 7A). The HRP concentration was lowered to 100-fold up to 0.25 μg/mL for further sensitivity analysis. Herein, the visible observation indicated that the color development plateaued at 25 μM of H₂O₂ and at 0.25 μg/mL of HRP concentration (FIG. 7B). ImageJ software-based analysis measured the color intensity of the silk fibroin mats, indicating the color development even at the lowest concentration (FIGS. 7C and D).

In Vivo Cutaneous Wounds Testing for Visible Sensing of Oxidative Stress

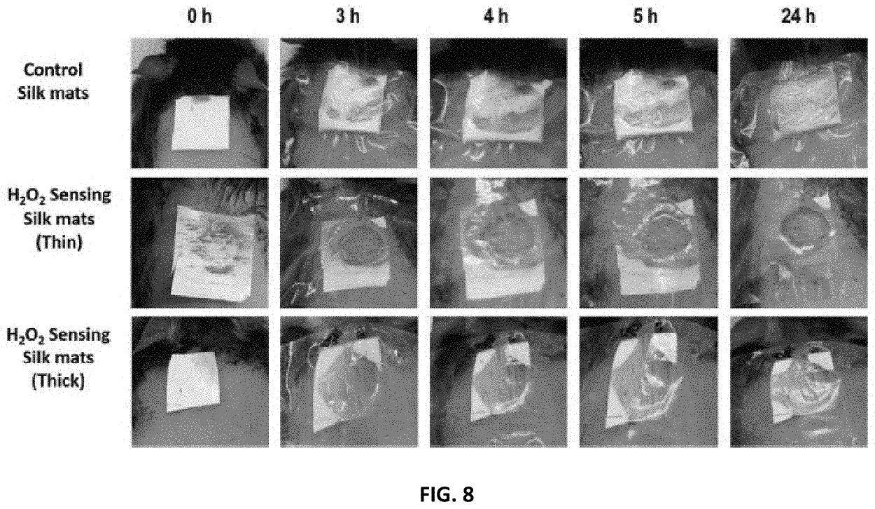

Visible color development into the Amplex red infused silk fibroin mats were observed, verifying the development of a color sensing mat for reactive oxygen species. Further, in order to evaluate the change in color of these sensing mats in the wound directly, the inventors applied the Amplex infused nanofibrous silk fibroin mats, both thin and thick, on diabetic mouse wounds. In terms of thickness, the silk fibroin nanofibrous thin mats were 0.001 mm thick, while the silk fibroin nanofibrous thick mats were 0.0043 mm thick, indicating a 4-fold difference in terms of thickness among both mats. While these thicknesses are used exemplary herein, other thicknesses are contemplated including from about 0.001 mm to about 1 mm in thickness, including all intervening thicknesses. These silk fibroin mats including control mats were placed onto the wounds and observed for 24 hours a for visible ROS sensing and color change. The Amplex infused silk fibroin mats were found to be absorbing the wound exudates and developed the visible pink color due to ROS sensing over a period of 24 hours, indicating its effectivity and sensitivity as compared to control mats which remained colorless. The thin mats were more pronounced in color development (FIG. 8). This might be due to the fact that these thin mats are almost 4-fold less dense than thick mats and have gotten saturated with the wound exudates containing peroxidase and H₂O₂, leading to the faster oxidation of Amplex red into resorufin and yielding purple color.

The wound release exudates include a variety of enzymes like peroxidases and biochemicals including oxidative stress inducing factors like H₂O₂, OH, and others. This is where the Amplex incorporated into the silk fibroin mat gets oxidized into visible resorufin and gets detected. Overall the level of visible detection of Amplex infused silk fibroin mats was accurate even at low concentration. However, this is indicated for the current mL of 5 mg/mL of Amplex infused into the nanofibrous silk fibroin mats. The inventors determine the Amplex loading capacities onto these nanofibrous silk mat and identify its sensitivity in H₂O₂ detection.

Material and Methods

Preparation of Silk Fibroin

The preparation of silk fibroin solution was performed according to the earlier reported protocol ion Rockwood et al. 2011, incorporated herein in its entirety. In detail, silk cocoons, isolated from Bombyx mori silkworms (Technical grade, Aurora Silk, USA) were weighted 5 g, cut into small dime sizes and were degummed using boiling water for 30 minutes containing 0.02M Na₂CO₃. The degummed silk was washed thrice for 20 minutes each and left further dried out completely. Lithium bromide (LiBr) solution of 9.3M was prepared, and the dried and degummed silk was incubated in oven at 60° C. for 4 hours, to prepare the silk fibroin solution. Post incubation, an amber colored and highly viscous silk fibroin solution is obtained. This solution is further dialyzed using ultrapure water for 48 hours. Further, this silk fibroin solution is centrifuged at 9000 rpm to remove the impurities. Finally, a clear and amber colored aqueous silk solution is obtained with average silk fibroin concentration ranging from 7-8% (w/v) and stored at 4° C. for further experimental use.

Fabrication of Nanofibrous Silk Mat Using Electrospinning

The clear and amber-colored silk fibroin solution collected from the previous preparation of silk fibroin step was used to further fabricate nanofibrous mats using electrospinning methodology. About 5 mL of silk solution was taken into a clean glass vial and 1 mL of 5% polyethylene oxide solution (PEO) was mixed into it under the mild stirring condition for 15 minutes. This silk solution was drawn in a 5 mL syringe attached to 23G needle and mounted to the syringe pump unit. The electrospinning unit was grounded with positive voltage lead connected to the solution containing syringe needle and the ground lead to the collector surface. The flow rate was adjusted to 1 mL/hr and the current was set to 2 A and electric potential to 20 kV. The distance between the syringe needle and the collector drum was set to 10 cm apart. Electrospinning of silk mat was performed until a visible mat of suitable thickness (at least 0.001 mm in thickness) was collected onto the collector unit. The silk fibroin nanofibrous mats synthesized here were used as control mats for the experiments.

Fabrication of Amplex Red Infused Nanofibrous Silk Mat Using Electrospinning

Amplex red infused nanofibrous silk mats were also synthesized using the electrospinning technique. In this process, the stock of Amplex red solution was prepared in DMSO firstly at a concentration of 5 mg/mL and mixed thoroughly. From this stock preparation, 1 ml of Amplex red solution was added to the 45 ml of silk solution (silk concentration=7-8% (w/v)) under mild stirring condition in increment of 200 μl every 5 minutes. This Amplex red-silk fibroin solution was stored under 4° C. and used for electrospinning of nanofibrous silk fibroin mats. This Amplex red-silk fibroin solution was drawn in a 5 mL syringe attached to 23G needle and mounted to the syringe pump unit. The electrospinning unit was grounded with positive voltage lead connected to the solution containing syringe needle and the ground lead to the collector surface. The flow rate was adjusted to 1 mL/hr and the current was set to 2 A and electric potential to 20 kV. The distance between the syringe needle and the collector drum was set to 10 cm apart. Electrospinning of silk mat was performed until a visible mat of suitable thickness was collected onto the collector unit.

Characterization of Silk Solution

Dynamic Light Scattering (DLS) and Zeta surface charge measurements were also acquired for the solubilized nanofibrous silk solution used for synthesizing electrospun nanofibrous silk mat using the Zetasizer Nano ZS and Zetasizer Nano software from Malvern Panalytical Ltd (Malvern, UK). This technique was used to characterize the diluted silk solution and find the particle size distribution of the silk fibers within the solution. 100 μL of 10× diluted silk solution was used for specifying the material and its property. An average of three separate measurements was obtained and used to calculate the values. Zeta potential analysis was also measured using the Zetasizer Nano ZS. Silk solution was diluted 10× in DI water and 1 mL was placed into a cuvette. An average of three separate measurements was referred and used to calculate the averages and standard deviations.

Surface Characterization of Silk Fibroin Nanofibrous Mats Synthesized with and without Amplex Red

The surface morphology of the nanofibrous silk fibroin mats and Amplex red infused nanofibrous silk fibroin mats were examined using scanning electron microscope (Zeiss ULTRA-55 FEG scanning electron microscope). For SEM imaging purposes, these mats were sputter-coated with a thin layer of gold and placed on imaging stub and recorded. To further characterize these nanofibrous silk fibroin mats, Fourier Transform Infrared Spectroscopy (FTIR) was also carried out using Perkin Elmer Spectrum-I instrument at room temperature in ATR mode from 4000-650 cm⁻¹. X-ray photoelectron spectroscopy (XPS) analysis was also conducted using an ESCALAB-250Xi spectrometer in an ultra-high vacuum chamber (below 7×10⁻⁹ mbar) using an Al-Kα monochromatic radiation source, operating at a power of 300 W (15 kV, 20 mA). Binding energies were calibrated based on C 1s peak at 284.8 eV±0.2 eV and the chemical functional groups were identified and deconvoluted using Thermofisher Avantage software.

In-Vitro Cellular Biocompatibility Analysis

Amplex Red compound was tested for in vitro cellular toxicity analysis against the human skin keratinocyte (HaCat) cells (purchased from ATCC, USA) using cell culture-based MTT assay. Herein, 10,000 cells were grown overnight into a 96 well plate using DMEM:F12 cell culture media. Different concentrations of Amplex red compound were prepared using incomplete cell culture media and incubated with HaCat cells for a time period of 24 hours and 48 hours respectively. Post incubation time period, MTT analysis was performed using the standard protocol described in Singh 2018, herein incorporated by reference into this disclosure in its entirety. (S. (Singh, A. Ly, S. Das, T. S. Sakthivel, S. Barkam and S. Seal, Artif. Cells, Nanomed., Biotechnol., 2018, 1-8). MTT assay compound (Thiazyolyl blue tetrazolium bromide) was then added for measuring the cellular viability, and absorbance was recorded for treated samples in comparison to control samples (HaCat cells only) and data analysis was performed.

Nanofibrous silk mats infused with Amplex Red were also tested for cellular biocompatibility. HaCat cells were grown onto these mats and imaged using the scanning electron microscope (Zeiss ULTRA-55 FEG scanning electron microscope). The cells were fixed using alcohol serial dilutions and subsequently sputter-coated with thin layer of gold just before the imaging. Cellular biocompatibility imaging was performed at a different resolution.

H₂O₂ Detection Assay and Limit of Detection (LOD) Analysis

Silk fibroin nanofibrous mats infused with or without Amplex red were tested for a visible change in color as a detection parameter of hydrogen peroxide. For this assay, phosphate buffer saline (PBS), horseradish peroxidase (HRP), and hydrogen peroxide (H₂O₂) solution were prepared. Horseradish peroxidase (HRP) stock solution was made by resuspending it to a concentration of 5 mg/mL, as per requirement. 3% H₂O₂ stock solution was diluted to make it a 1% H₂O₂ solution and placed in a conical tube wrapped in aluminum foil, avoiding direct light. Further dilutions of these H₂O₂ and HRP were made from these stocks as per requirement using PBS. The visible change in color reaction was optimized using 1 mL PBS and 100 μL 2.5 mg/mL HRP. The control reaction vial had 10 μL of Amplex red reagent (5 mg/mL) added to it. Once these vials were prepared, 100 μL 1% H₂O₂ were added to all vials and the visible change in color was recorded.

To determine the limit of detection (LOD), different concentrations of HRP and H₂O₂ were prepared. Control silk fibroin mats and Amplex red infused silk fibroin mats of similar sizes (about 1 cm×0.5 cm size) were placed in a 12-well plate. In each well, 1 mL of PBS, 50 μL of specified HRP concentration, and 50 μL of specified H₂O₂ concentration were added. These nanofibrous mats were imaged for visible change in color development at different time points and further analysis was carried out using ImageJ software.

Animals

All experimental protocols were approved by the Institutional Animal Care and Use Committee at University of Colorado Denver-Anschutz Medical Campus and followed the guidelines described in the NIH Guide for the Care and Use of Laboratory Animals. Age-matched, female, genetically diabetic C57BKS.Cg-m/Leprdb/J (Db/Db) mice were used in these experiments.

In Vivo Animal Experiments for Visible Change in Color Detection of H₂O₂

To examine the ability of silk fibroin nanofibrous mats infused with Amplex red to detect oxidative stress in vivo, 12-week-old Db/Db mice were anesthetized with inhaled isoflurane and shaved before wounding. The dorsal skin was sterilized with alcohol and Betadine (Purdue Pharma, Stamford, Conn.). Each mouse underwent a single, dorsal, full-thickness wound (including panniculus carnosum) with an 8 mm punch biopsy (Miltex Inc., York, Pa.). One set of the wounds was covered with Amplex infused silk fibroin nanofibrous mats and another set was covered with a control silk fibroin nanofibrous mats only. All wounds were then dressed with Tegaderm (3M, St Paul, Minn.), and pictures of the wounds were taken directly after the mats were applied, and at 3, 4, 5, and 24 hours post-application.

### Example 2—Prophetic Use in Human Wounds to Monitor Wound Healing

A 52 year old man presented with chronic wound sites of diabetic ulcers on both feet. An electrospun Amplex red infused nanofibrous silk fibroin mat infused with Amplex red was prepared as described in Example 1. The electrospun Amplex red infused nanofibrous mat was applied to the wound site and a color change was detected at a time period after application. The intensity of the color change indicated the presence of a large amount of hydrogen peroxide which is indicative of ROS. The electrospun Amplex red infused nanofibrous silk fibroin mat as removed and a therapeutic agent was applied to the wound site. The wound site was then covered with a new electropsun Amplex red infused nanofibrous silk fibroin mat. After a period of time, the new electropsun Amplex red infused nanofibrous silk fibroin mat was examined for a change in color. A change in color was observed and was compared to the change in color in the original electropsun Amplex red infused nanofibrous silk fibroin mat. The degree of color change in the new mat was significantly less than the original mat thus indicating the wound is healing. An additional application of the therapeutic agent is administered and the steps repeated until no color change is observed indicating the wound has healed.

### CONCLUSION

In conclusion, the inventors successfully fabricated nanofibrous silk fibroin mats through the processing of silk fibroin solution directly from the raw silk cocoons. These nanofibrous silk fibroin mats were infused with Amplex red dye for a real-time sensing of the oxidative stress in the wounds through the oxidation of Amplex into resorufin by H₂O₂ moiety. FTIR analysis confirmed the Amplex red infusion into these fine nanofibers and the chemical integrity into the mats post electrospinning. In vitro color sensing experiments indicated that concentrations of H₂O₂ as low as 25 μM with 0.25 μg/ml of HRP can easily produce a visible color change. In vitro cellular biocompatibility was also observed for the mats and further in vivo experiments were performed using the diabetic wounds, which indicated the visible color sensing mats after a 24 hour incubation time period, indicative of sensing oxidative stress in these wounds. These H₂O₂ sensing mats are ideal to monitor the changes in oxidative stress and ROS levels directly in the wounds and allow the adaptation and personalization of treatment based on the levels of oxidative stress of each wound.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

It will thus be seen that the objects set forth above, and those made apparent from the foregoing disclosure, are efficiently attained. Since certain changes may be made in the above construction without departing from the scope of the invention, it is intended that all matters contained in the foregoing disclosure or shown in the accompanying drawings shall be interpreted as illustrative and not in a limiting sense.

It is also to be understood that the following claims are intended to cover all of the generic and specific features of the invention herein disclosed, and all statements of the scope of the invention that, as a matter of language, might be said to fall therebetween. Now that the invention has been described,

What is claimed is:

1. A composition for detecting reactive oxygen species (ROS) at a wound site comprising:
an electrospun nanofibrous mat; and
a reactive oxygen species (ROS) reactive detectable reagent infused into the electrospun nanofibrous mat.

2. The composition of claim 1, wherein the nanofibers are silk.

3. The composition of claim 2, wherein the silk is obtained from silkworms.

4. The composition of claim 1, wherein the ROS reactive detectable reagent comprises an Amplex red compound.

5. The composition of claim 1, wherein the electrospun nanofibrous mat is a component of an adhesive bandage, wound dressing, surgical drape, or suture.

6. The composition of claim 1, wherein the electrospun fibrous mat is between about 0.001 mm to about 0.0043 mm in thickness.

7. A method of detecting reactive oxygen species (ROS) levels at a wound site of a patient in real time to determine the need for a dressing change comprising:
applying an electrospun nanofibrous mat infused with an ROS detectable reagent to the wound site of the patient; and
detecting a change in color of the electrospun nanofibrous mat;
wherein the change in color indicates presence of ROS.

8. The method of claim 7, wherein the nanofibers are silk.

9. The method of claim 7, wherein the ROS detectable reagent comprises an Amplex red compound.

10. The method of claim 7, further comprising comparing degree of color change in the electropsun nanofibrous mat to a control to measure an amount of ROS at the wound site of the patient.

11. The method of claim 10, wherein intensity of the degree of color change indicates a high amount of the ROS at the wound site.

12. The method of claim 11, further comprising administering a therapeutically effective amount of a therapeutic agent to the wound site if the degree of color change is intense.

13. The method of claim 7, wherein the electrospun nanofibrous mat is a component of an adhesive bandage, wound dressing, surgical drape, or suture.

14. A method of monitoring healing of a wound site in a patient comprising:
applying a first electrospun nanofibrous mat infused with an ROS detectable reagent to the wound site of the patient;
detecting a change in color of the first electrospun nanofibrous mat;
removing the first electrospun nanofibrous mat from the wound site;
applying a second electrospun nanofibrous mat infused with an ROS detectable reagent to the wound site of the patient;
detecting a change in color of the second electropsun nanofibrous mat; and
comparing the change in color of the second electrospun nanofibrous mat to the change in color of the first electrospun nanofibrous mat;
wherein if a degree of the change in color of the second electrospun nanofibrous mat is less than the change in color of the first electrospun nanofibrous mat then the wound site is healing.

15. The method of claim 14, further comprising administering a therapeutic agent to the wound site prior to applying the second electrospun nanofibrous mat.

16. The method of claim 14, wherein the nanofibers are silk.

17. The method of claim 14, wherein the ROS detectable reagent comprises an Amplex red compound.

18. The method of claim 14, wherein the electrospun nanofibrous mat is a component of an adhesive bandage, wound dressing, surgical drape, or suture.
